(19) United States
(12) Patent Application Publication
(10) Pub. No.: US 2024/0122696 A1
(43) Pub. Date: Apr. 18, 2024

(54) Method and Device for Ligament Reconstruction

(71) Applicants: Shirin Jamali, Oakland, CA (US); Amir A. Jamali, Oakland, CA (US)

(72) Inventors: Shirin Jamali, Oakland, CA (US); Amir A. Jamali, Oakland, CA (US)

(21) Appl. No.: 18/487,441

(22) Filed: Oct. 16, 2023

Related U.S. Application Data
(60) Provisional application No. 63/379,592, filed on Oct. 14, 2022.

(51) Int. Cl. A61F 2/08; A61B 17/04; A61B 17/06

(52) U.S. Cl. CPC A61F 2/0811; A61B 17/0401; A61B 17/0466; A61B 17/06004; A61B 2017/0404; A61B 2017/0409; A61B 2017/0464; A61F 2002/0817; A61F 2002/0858; A61F 2002/087

(57) ABSTRACT

A device for stabilizing a soft tissue graft to a fixation device for ligament reconstruction, including a generally planar and rectangular elongate pad of woven suture material having a proximal portion with an upper edge and upper corners and a distal portion having a bottom edge, wherein said proximal portion transitions into two integral, generally cylindrical suture limbs extending from the upper corners, each of which transition into and terminate in a distal end configured to fit through the eye of a surgical needle. A method of attaching the device to a tissue graft is also described. The device facilitates rapid fixation of the soft tissue graft in such a way as to avoid creep and laxity of the final construct.

### CROSS REFERENCES TO RELATED APPLICATIONS

The present application claims the benefit of the filing date of U.S. Provisional Patent Application Ser. No. 63/379,592, filed Oct. 14, 2022 (Oct. 14, 2022), which application is incorporated in its entirety by reference herein.

### BACKGROUND OF THE INVENTION

Field of the Invention: The present invention relates most generally to the field of ligament reconstruction.

Background Discussion: Ligament injuries are some of the more common sports injuries encountered by physicians. Ligament injuries can be reconstructed with a native autologous graft obtained from an adjacent area of a patient or through the use of a cadaveric graft, known as an allograft. Many methods have been used to achieve fixation of these grafts to the native bone. These include screws, pins, staples, and buttons, most of which are made of metal or polymers and which engage the graft to approximate and secure it to the surface of the bone. Many ligament grafts are placed into the joint by drilling bone tunnels into which the ligament is passed. Subsequently the ligament graft is held in place with a large screw to compress it against the wall of the tunnel. This is called an interference screw. Another option for fixation is to tie sutures to the ligament graft and have those sutures exiting the ends of the graft. The sutures are then passed through a hole on the outer surface of the bone and tied or locked onto a round or often rectangular button, holding the graft rigidly in placed within the bone tunnel. This button technique requires rigid fixation of the grafts to a suture from the button or to the button itself. Most buttons are disposed with a suture loop passing through the fenestrations that they contain. The suture loop is either of fixed or of adjustable length depending on the design of the button. The graft is passed through the suture loop or sutured to the suture loop depending on surgeon preference.

One of the challenges when using a soft tissue graft with a button device is in providing adequate stabilization of the graft to the button suture loop without cut-out or laxity in the graft construct caused by a gradual creep or pulling away of the graft from the button during tensioning of the graft or during the patient's recovery. The present invention addresses this by first providing a device for effecting a sandwich technique using a woven suture pad with two extensions or limbs of suture extending from one end of the pad, and then providing a method by which to attach the inventive device to a soft tissue graft for use in a ligament reconstruction.

### BRIEF SUMMARY OF THE INVENTION

The present invention is a device for stabilizing a soft tissue graft to a fixation device (such as a metal button) for ligament reconstruction. The device includes a generally planar and rectangular elongate pad of woven suture material having a proximal portion with an upper edge and upper corners and a distal portion having a bottom edge, wherein said proximal portion transitions into two integral, generally cylindrical suture limbs extending from the upper corners, each of which transition into and terminates in a distal end configured to fit through the eye of a surgical needle or such that the two suture limbs are fused into one woven limb at the most proximal aspect or such that the two suture limbs are fused into a solitary needle at the most proximal aspect. A method of attaching the device to a tissue graft is also disclosed. The device facilitates rapid fixation of the soft tissue graft in such a way as to avoid creep and laxity of the final construct.

### BRIEF DESCRIPTION OF SEVERAL VIEWS OF THE DRAWINGS

An understanding of the invention and its various objects and advantages are apparent when consideration is also given to the following detailed description thereof. Such description makes reference to the annexed drawings wherein:

FIG. 1 is an anterior view of a soft tissue graft such as a quadriceps tendon graft or Achilles tendon graft;

FIG. 2 is a front view of the inventive fixation device showing a rectangular pad of suture and two extending lines of suture;

FIG. 3 is an anterior view of the soft tissue graft during placement of the fixation device on and around the graft;

FIG. 4 is the same anterior view of the soft tissue graft after one pass of the sutures through the distal portion of the rectangular pad of the fixation device, then through the graft, and then once again through the proximal portion of the rectangular pad of the fixation device;

FIG. 5 is again the same anterior view of the soft tissue graft after the sutures of the fixation device are brought back to the front of the tissue portion of the construct, slightly further up in relation to the view of FIG. 4, and here seen passed through the fixation device, then through the graft, and then once again through the fixation device;

FIG. 6 is the same view showing the sutures coming out from the back side of the combined construct;

FIG. 7 is the same view after the sutures of the fixation device are brought back to the front of the construct for another pass to through the front of the construct, again slightly further up and are again passed through the fixation device, then through the graft, and then once again through the fixation device;

FIG. 8 is the same view showing the sutures again emerging from the back of the combined construct;

FIG. 9 is again the same view showing the sutures brought back to the front of the construct for still another pass to the front of the construct, again slightly further up and are again passed through the fixation device, then through the graft, and then once again through the fixation device;

FIG. 10 is the same view sutures emerging from the back of the combined construct as the sutures stack in spaced apart rows to comprise a suture wrap through and around the graft;

FIG. 11 is the same view after one line of the suture loop has been cut from the needle and passed through the graft separately from the other line;

FIG. 12 is an anterior view of the soft tissue graft after the two ends of the suture have been tied together around the graft;

FIG. 13 is an anterior view of the fixation device and graft with a metal button and a closed suture loop fixed to the graft with the fixation device;

FIG. 14 is an anterior view of the fixation device and graft with a metal button and an adjustable suture loop fixed to the graft with the fixation device; and

FIG. 15 is a proximally directed view of the knee during ACL reconstruction showing the graft, the fixation device, and the adjustable loop button on the femoral side in their respective final positions during surgery.

### DETAILED DESCRIPTION OF THE INVENTION

The fixation device described herein first includes a pad of woven surgical suture generally configured as a generally planar elongate rectangle (hereinafter referred to as a “suture pad”). The suture pad, in turn, includes a proximal portion and a distal portion, the former having two integral lengths or lines of suture extending from each corner of that portion. The distal ends of the two lengths of suture (hereinafter termed “suture limbs”) can be combined and together fed through the eye of a needle for use in attaching the suture pad to a soft tissue graft. Alternatively, the distal ends of the suture limbs may each be separately fed through the eyes of separate needles and thus disposed with a dedicated needle. In yet another embodiment, the two sutures can be fused into one suture which is then attached to a needle or the two sutures can be independently attached to the same needle without an eyelet. The fixation device is configured to enable it to be wrapped around a soft tissue graft; it does so by circumferentially attaching to a graft end using the above-described suture limbs extending from the corners of the proximal portion of the suture pad. The suture pad is first draped over an end of the graft with the proximal portion of the suture pad disposed on the rear (posterior) side of the graft and the distal portion on the front (anterior) side of the graft, and the two suture limbs are then brought from the rear of the graft to the front of the graft either by passing them under the graft axilla (in the event the graft has an axilla dividing the distal portion of the graft into two bundles of fibrous connective tissue) or by passing the needle(s) through the graft below the proximal and distal portions of the suture pad. The needle and suture limbs are then passed through the distal portion of the suture pad using a needle, through the graft, and then through the proximal portion of the suture pad. In most instances, this general pattern is substantially repeated to create a succession of stacked levels of suture limbs, each circumferentially binding the fixation device more securely to the graft end. The passes after the first pass, however, depart slightly from the first pass steps as they involve an initial step of pulling the needle and suture limbs over the top of the graft, draping the suture limbs around the sides of the graft, and then again passing the needle and suture limbs front-to-rear at a slightly higher level than the preceding pass. In such a way, the fixation device achieves robust fixation to the graft.

The fixation device can be preferentially sutured around the graft in such a way as to hold any number of standard surgical tools for fixation of a ligament, e.g., the anterior cruciate ligament, such tools including open-loop buttons, closed-loop buttons, or other methods of cortical fixation. In this way, rapid fixation of the fixation buttons to the graft can be achieved while maintaining secure fixation to avoid complications of loosening of the graft over time.

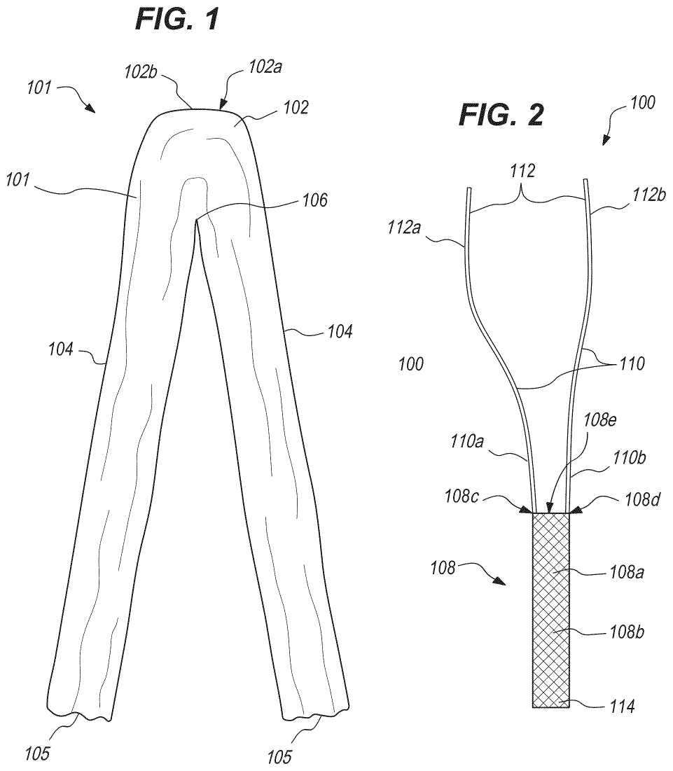

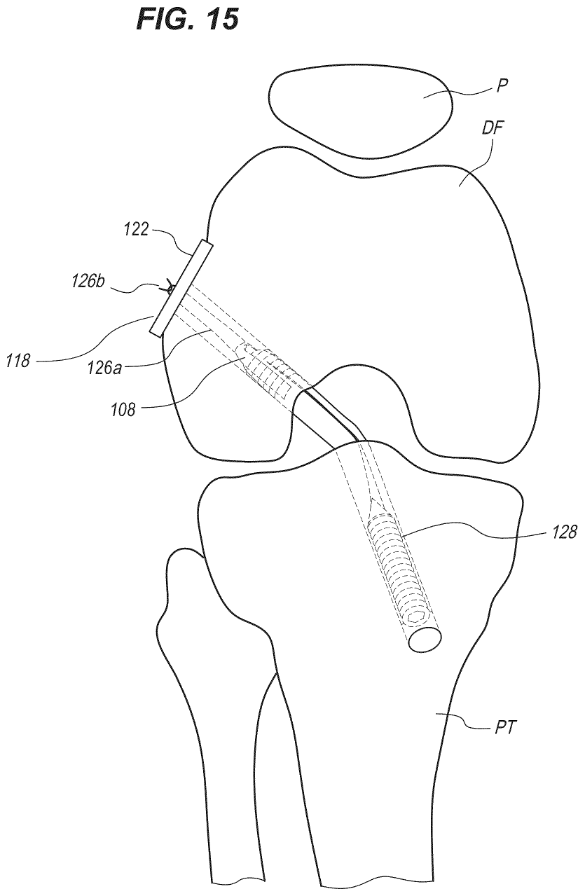

FIG. 1 is an anterior view of a soft tissue graft such as a quadriceps tendon or Achilles tendon. The superior (proximal) portion 102 of the graft 101 has a proximal end 102a having a generally straight upper edge 102b. In some procedures, the lower part of the graft may be divided into two elongate distal bundles 104, converging at an axilla or crotch 106 and having inferior ends 105. The thickness of the graft is a function of the donor anatomy, though when viewed on end, the graft is generally elliptical in shape. [It should be noted that a quadriceps tendon harvested for a graft often includes an axilla where the tendon divides into two bundles of tissue, but the graft for the present invention need not have an axilla or other division into bundles. Thus, while a bifurcated graft is described herein for illustrative purposes, the method steps and inventive surgical technique can be carried out using a unitary graft with no bifurcation or division into bundles. Use of an undivided graft is shown in FIG. 15.]

Turning next to FIG. 2, there is shown an anterior view of the fixation device 100. In an embodiment, the device includes a panel of woven suture material of varying configuration and reticulation density creating a generally elongate rectangular lower portion 108, which in embodiments takes the form of a woven fabric strap, referred to herein as a suture pad. The suture pad 108 can be made using any of a number of well-known weaving patterns, including plain, twill, satin, basket, leno, mock leno, etc. It can also be made with variations in porosity to allow adequate contact between the graft and the native bone to facilitate biological incorporation. It may be roughly divided into an upper, proximal portion 108a and a lower, distal portion 108b, each comprising roughly half the total interior region defined by the suture pad.

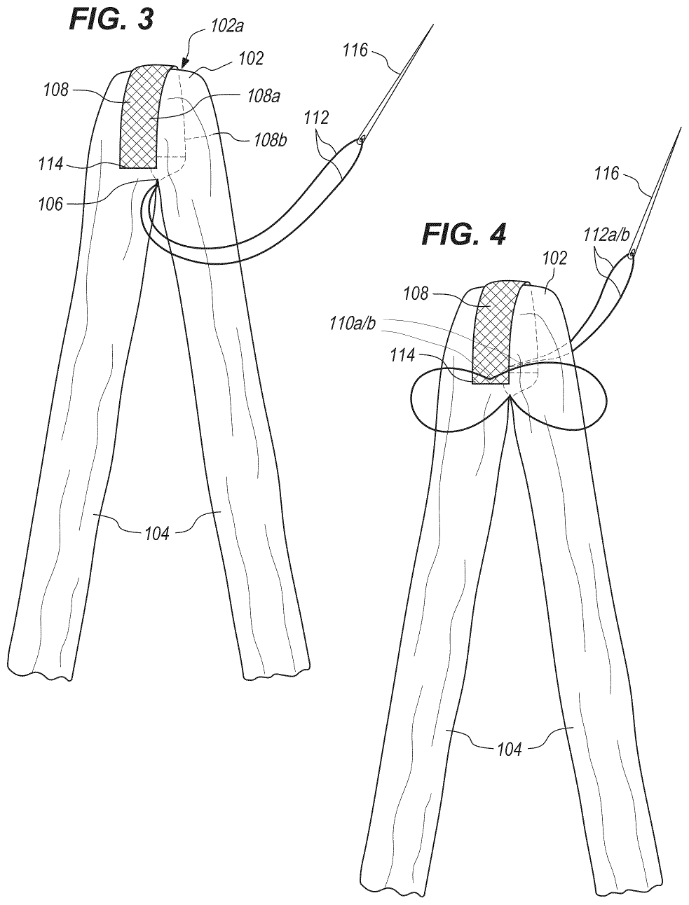

The upper portion 108a of the suture pad 108 transitions to two integral and generally cylindrical upper portions of suture 110a/110b extending from the upper corners 108c, 108d of the suture pad on its upper edge 108e and formed into two unitary braided or twisted singular cylindrical sutures. In turn, the cylindrical upper portions of sutures 110a/100b (hereinafter “suture limbs”) each transition and may substantially reduce in diameter to terminate in a distal end 112a/112b of either a single length of flat or round suture or remain in the same form, sized with dimensions (whether flat or round) to fit through the eye of a single needle 116 (FIG. 3) or into two needles (not shown). Alternatively, the suture limbs can be fused into a single needle base or woven into a single suture which is in turn fused to the base of a single needle (not shown). The bottom end 114 of the distal portion 108b of the suture pad is a generally straight edge.

FIG. 3 is an anterior view of the soft tissue graft with its unitary upper portion 102 and its bifurcated lower portion with two bundles or legs 104. Here it can be seen that the suture pad 108 is folded generally in half over the proximal end 102a of the upper portion 102 of the graft 101, such that the proximal portion 108a of the suture pad and the distal portion 108b of the suture pad are disposed on opposing (front and rear) sides of the graft, and the distal portion of the suture pad 108b is positioned so that its bottom edge 114 closely approximates the graft crotch 106. Placement of the fixation device in this manner is the first step in practicing the inventive method of forming the combined graft and fixation device (i.e., the “combined construct”).

The succeeding steps (those following the above-described first step) are illustrated and described in connection with FIGS. 3-15, wherein, and returning now to FIG. 3, after the suture pad is draped over the top of the graft, the top portion of the suture limbs 110a/110b are passed under the axilla 106, with their respective distal ends 112a/112b passed through a needle 116, which is thereafter used for passing the sutures through both the suture pad (both proximal 108a and distal portions 108b) and the graft 101. Alternatively, when using a unitary graft having no division into bundles, the needle is first passed through the graft itself, initially penetrating the graft below the top of the suture pad on the rear side and emerging below the bottom end 114 of the suture pad on the front side of the combined construct, such that the suture limbs are positioned similarly to the position illustrated in FIG. 3. Either graft configuration is entirely suitable for the inventive method described herein insofar as each position the needle for the first in a series of front-to-rear-side passes through the distal portion of the suture pad, the graft, and the proximal portion of the suture pad.

Looking next at FIG. 4, there is shown an anterior view of the soft tissue graft 101 with its proximal segment 102, distal bundles 104, and the fixation device 100 again positioned for a second pass through the combined construct. The distal ends 112a/112b of the suture limbs 110a/110b have been passed through the distal portion 108b of the suture pad and the upper portion 102 of the graft 101 and have emerged from the rear of the graft.

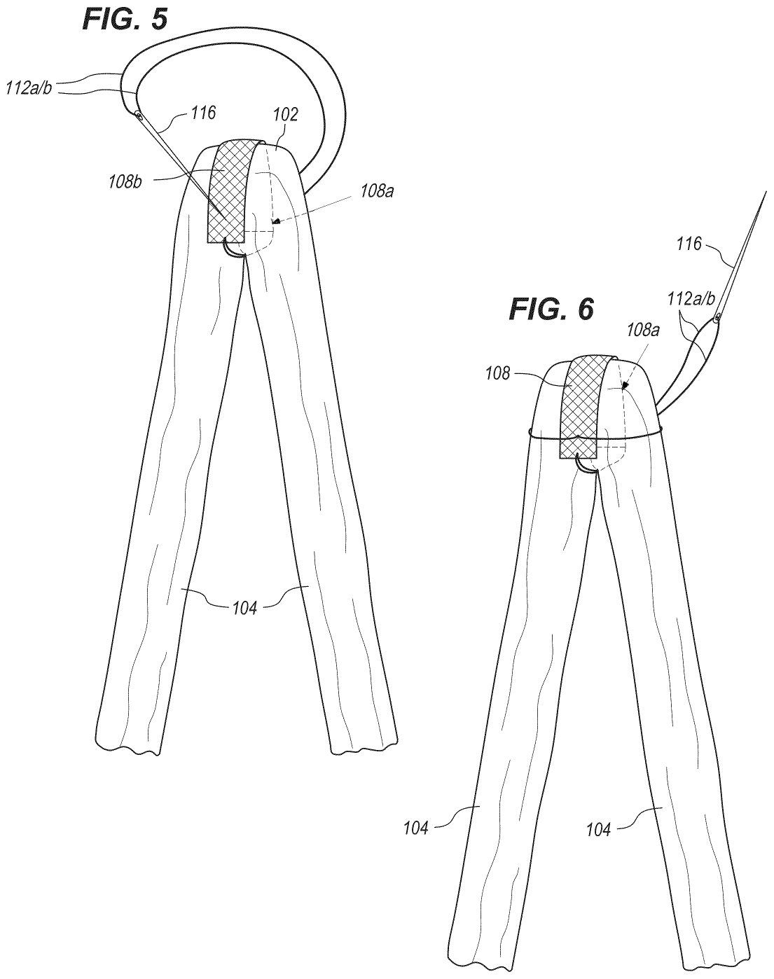

FIG. 5 is an anterior view of the developing combined construct, including the soft tissue graft 101 with its proximal segment 102, distal legs 104, this view showing needle 116 being passed once again from the front (anterior) side of the construct, and thus also the distal portion 108b of the fixation device, and into the graft itself.

FIG. 6 is an anterior view of the combined construct after passage of the needle from front to rear with the two suture limbs 110a/110b pulled into loops that wrap around the sides of the proximal portion 102 of the graft. Thus, the needle has here made a second pass through the distal portion 108b of the fixation device 100 with the distal ends of the sutures 112a/112b pulled through and captured by the eye of a single needle 116.

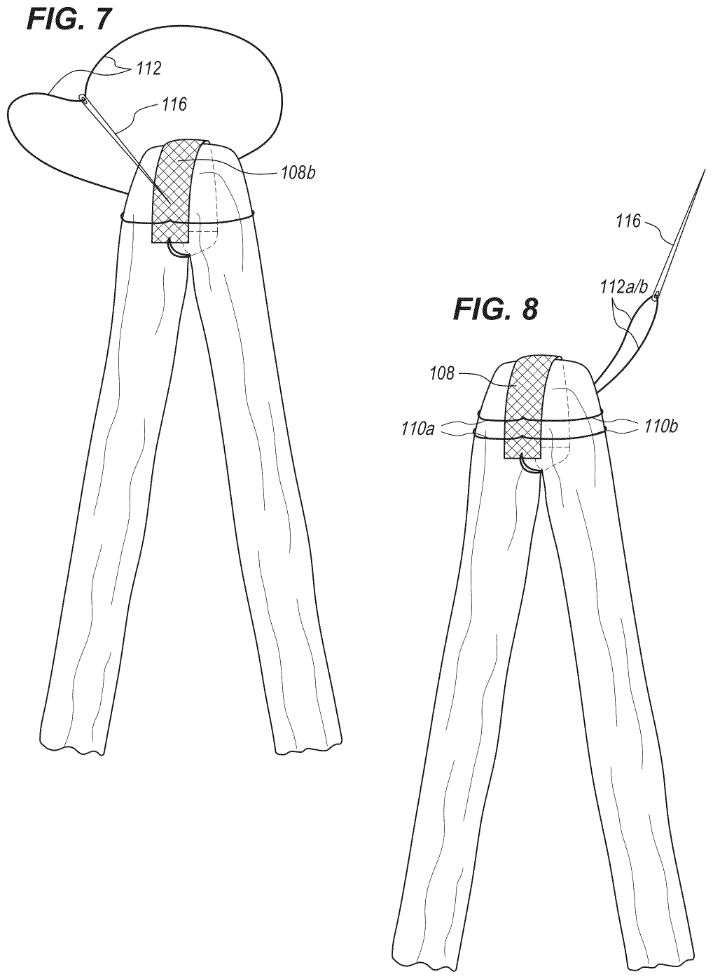

FIG. 7 is an anterior view of the soft tissue graft preliminary to a third pass of the needle from anterior to posterior through the combined construct, wherein both suture limbs 110a/110b are draped around the graft as the needle is moved over the top of the graft and positioned on the front side of the combined construct so that it can again be inserted into and then pulled through the distal portion 108b of the suture pad 108, then the proximal portion 102 of the graft 101, and then the proximal portion 108a of the suture pad. The repetition of these steps to create a plurality of spaced-apart and stacked suture loops surrounding and passing through the proximal portion of the graft is shown in FIGS. 8-10.

FIG. 8 is an anterior view of the soft tissue graft after the passage of the third loop of suture with the needle 116 and sutures 110a/110b having passed through both the distal and proximal portions of the suture pad and the graft.

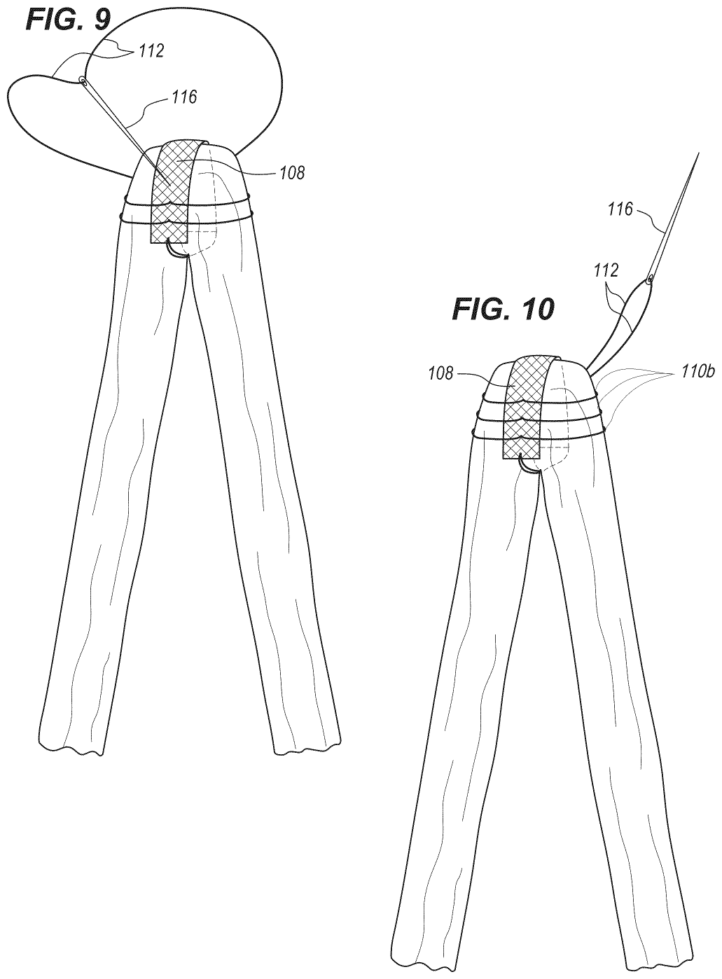

FIG. 9 is an anterior view of the soft tissue graft with the needle poised for a fourth pass from front to rear of the combined construct, again first involving draping both suture limbs 110a/110b about and around the sides of the graft as the needle is moved over the graft and positioned for penetration through the front side of the fixation device at the distal portion 108b, then the graft, and then the proximal portion 108a of the suture pad.

FIG. 10 is an anterior view of the soft tissue graft after the passage of the fourth loop of suture with the needle and sutures have been passed through the suture pad and the graft.

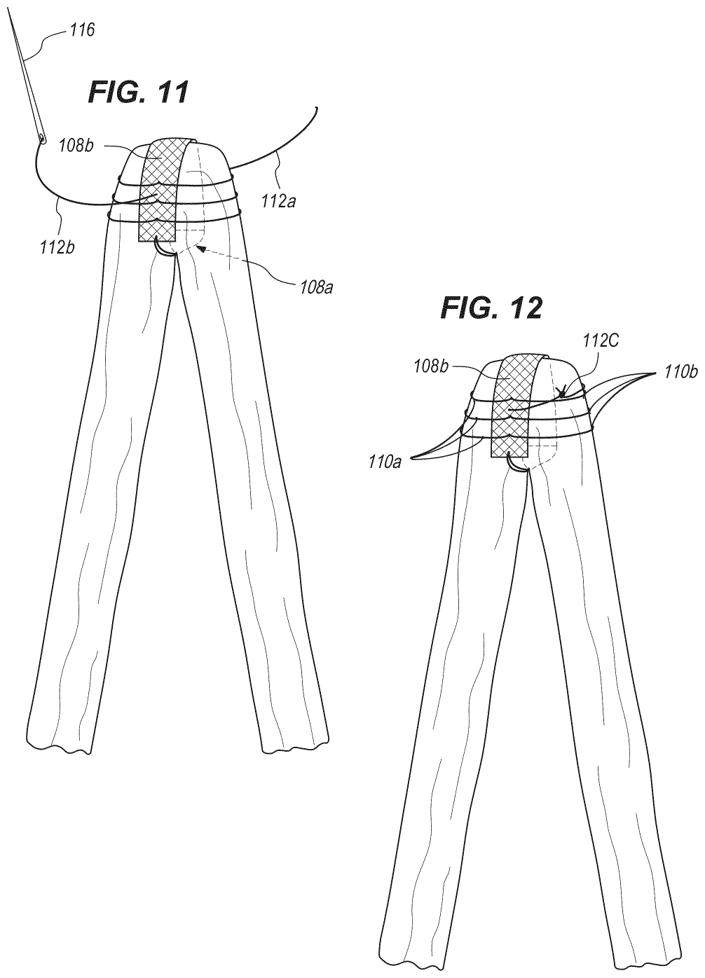

FIG. 11 is an anterior view of the combined construct after one suture limb 110b has been cut and separated from the needle 116 while the other suture limb 110a remains attached to the needle. The needle and the attached suture limb are then passed through the combined construct rear-to-front, first through the proximal portion 108a of the suture pad, then through the graft, and then through the distal portion of the suture pad to the front side of the combined construct.

FIG. 12 is an anterior view of the soft tissue graft, after the two distal ends 112a/112b of the suture limbs have been tied together in a knot 112c, thus completing a final loop around one side of the graft.

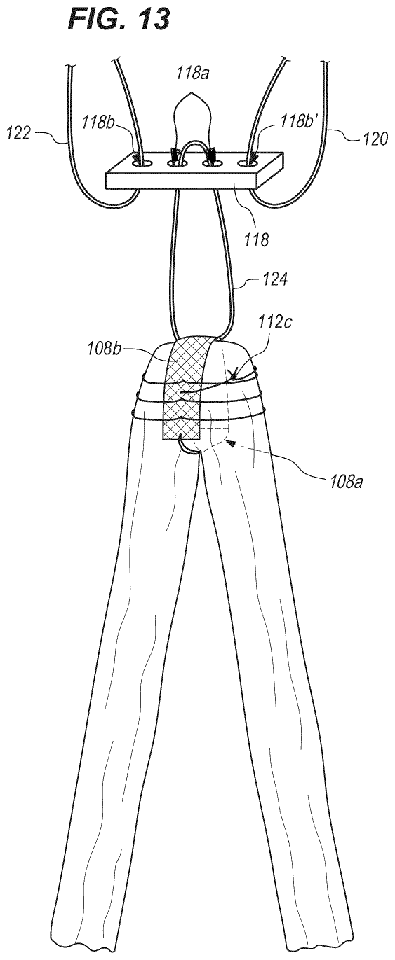

FIG. 13 is an anterior view of the combined construct (shown here with the fixation device securely attached to the graft) connected to a metal button 118 having inboard holes 118a and outboard holes 118b/118b′, linearly aligned along a longitudinal axis of the button, and further showing a leading suture limb 120, and a lagging suture limb 122 each operatively looped through one of the outboard holes, 118b and 118b′, respectively. The metal button in this instance is attached to a closed suture loop 124 that has been fixed to the combined construct by suturing the suture pad 108 of the fixation device over the closed suture loop 124 of the button. (This step is carried out before attaching the fixation device to the graft, as shown in FIGS. 3-11, above.)

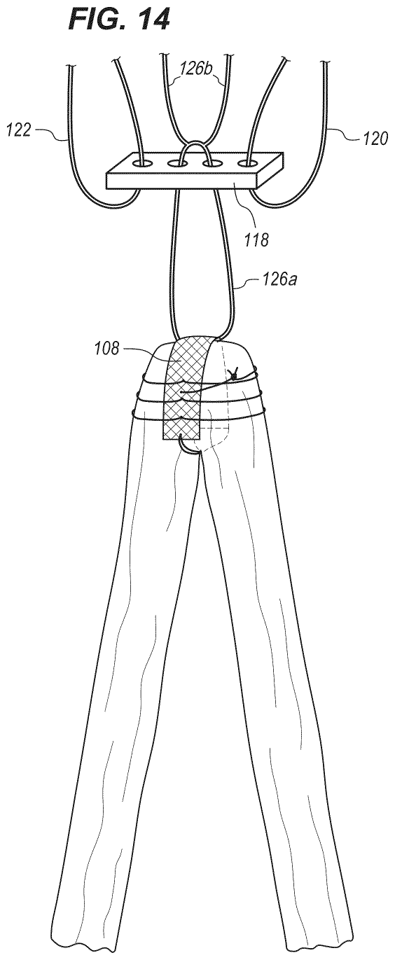

FIG. 14 is an anterior view of the fixation device with a metal button 118 with a leading suture limb 120, and a lagging suture limb 122. The metal button in this case is attached to an adjustable suture loop 126 that has been coupled to the graft in the same manner as the closed suture loop in FIG. 13. The adjustable suture loop has a lower portion 126a and an upper portion 126b, and the length of the lower portion 126a of the adjustable closed suture loop below the button 118 can be decreased by pulling (upwardly and away from the button) the ends of the upper portion 126b of the adjustable closed suture loop.

FIG. 15 is a proximally directed view of a human knee during ACL reconstruction. The distal femur DF is shown, along with the patella P, and the proximal tibia PT. An anatomical reconstruction of the anterior cruciate ligament is performed by making tunnels for both the femoral and tibial portions of the ligament in the bone. On the femoral side, the cortex (cortical portion of bone) is maintained, and the metal cortical button 118 with an adjustable closed suture loop 126 is passed after fixing the lower portion of the adjustable loop 126a to the graft using the fixation device 100. On the tibial side, a number of fixation techniques, including an interference screw 128, can be used.

The above disclosure will enable one of ordinary skill in the art to practice the invention. While this disclosure describes various embodiments of the invention, these embodiments do not limit the invention to the exact construction, apparent dimensional relationships, and the operation shown and described. Modifications, alternative constructions, changes, and equivalents will readily occur to those skilled in the art and may be employed, as suitable, without departing from the true spirit and scope of the invention.

Therefore, the above description and illustrations should not be construed as limiting the scope of the invention, which will be defined by claims set out in a non-provisional patent application claiming the benefit of the filing date of the instant application.

What is claimed as invention is:

1. A fixation device for ligament reconstruction, comprising a generally planar rectangular pad of suture material (herein “suture pad”) having a proximal portion with upper corners and a distal portion having a bottom edge, two cylindrical upper portions of suture (herein “suture limbs”), one each extending from one of said upper corners, each of said suture limbs transitioning into and terminating in a distal end configured to fit connect to a surgical needle.

2. The fixation device of claim 1, wherein said suture pad is fabricated from woven suture.

3. The fixation device of claim 1, wherein said suture limbs are integral with suture material extending from said suture pad.

4. The fixation device of claim 1, wherein said suture pad is elongate.

5. The fixation device of claim 1, wherein said bottom edge of said distal portion of said suture pad is generally straight.

6. A method of preparing a soft tissue graft for use in ligament reconstruction, comprising the steps of:
(a) providing a soft tissue graft having a front side, a rear side, an upper portion having an upper end, and a lower portion;
(b) providing a fixation device having a generally planar rectangular pad of suture material (herein “suture pad”) having a proximal portion with upper corners and a distal portion having a bottom edge, two cylindrical upper portions of suture (herein “suture limbs”), one each extending from one of said upper corners, each of said suture limbs transitioning into and terminating in a distal end configured to connect to a surgical needle;
(c) providing at least one surgical needle;
(d) connecting the distal ends of the suture limbs or separately to one or two needles, respectively;
(e) draping the suture pad over the end of the upper portion of the graft such that the proximal portion of the suture pad is approximated to the rear side of the upper portion of the graft and the distal portion of the suture pad is approximated to the front side of the upper portion of the graft;
(f) passing the suture limbs from the rear side of the graft to the front side of the graft and thereafter passing the needle or needles and suture limbs through the distal portion of the suture pad, through the graft, and then through the proximal portion of the suture pad;
(g) passing the needle or needles and suture limbs over the graft while separating the suture limbs such that together they generally circumferentially surround the upper portion of the graft;
(h) passing the needle or needles and suture limbs from the front side of the graft to the rear side of the graft by inserting the needle or needles and suture limbs through the distal portion of the suture pad slightly higher (more proximal) than the immediately preceding point of needle penetration, through the graft, and through the proximal portion of the suture pad;
(i) repeating steps (g) and (h) until a plurality of spaced-apart and stacked suture loops surround and pass through the proximal portion of the graft to provide a secure attachment of the fixation device to the graft and to create a combined construct;
(j) removing the needle or needles from the suture limbs; and
(k) connecting the combined construct to a surgical tool for fixation of a ligament.

7. The method of claim 6, wherein the soft tissue graft is an autologous graft obtained from an adjacent area of a patient.

8. The method of claim 6, wherein the soft tissue graft is a cadaveric graft.

9. The method of claim 6, wherein the soft tissue graft is a quadriceps tendon.

10. The method of claim 6, wherein the soft tissue graft is an Achilles tendon.

11. The method of claim 6, wherein the upper portion of the soft tissue graft is a unitary portion and the lower portion includes two fibrous bundles that converge at the unitary upper portion at an axilla, and wherein the first step of passing the needle and suture limbs from the rears side of the graft to the front side of the graft entails passing the needle and suture limbs under the graft axilla.

12. The method of claim 6, further including a step following step (k) wherein the distal ends of the suture limbs are tied with a knot.

13. The method of claim 6, where the surgical tool is either an open-loop button or a closed-loop button.

14. The method of claim 13, further including the step of connecting the suture pad to the button before draping it over the upper end of the upper portion of the soft tissue graft.
